(19) United States
(12) Patent Application Publication
(10) Pub. No.: US 2006/0073188 A1
(43) Pub. Date: Apr. 6, 2006

(54) Fast-dissolving isotropic expanded microporous composition or structure for pharmaceutical, veterinary, dietetic, food or cosmetic use and method for obtaining same

(75) Inventors: Eric Goutay, Lauzerville (FR); Laurence Lachamp, Clermont-Ferrand (FR); Jacques Frances, Toulouse (FR); Joel Bougaret, Lanta (FR); Bruno Paillard, Clermont-Ferrand (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: PIERRE FABRE MEDICAMENT, BOULOGNE-BILLANCOURT (FR)

(21) Appl. No.: 11/287,070

(22) Filed: Nov. 25, 2005

Related U.S. Application Data
(63) Continuation-in-part of application No. 09/937,678, filed on Sep. 28, 2001.

(30) Foreign Application Priority Data
Mar. 31, 1999 (FR) FR 99/04,033

(51) Int. Cl. A61K 47/00

(52) U.S. Cl. 424/439

(57) ABSTRACT

The invention relates to novel fast-disintegrating, or even instant-disintegrating, homogeneous microporous compositions for pharmaceutical, veterinary, food, dietetic or cosmetic use, intended for the oral route or to be applied in contact with the mucous membranes and a method for producing them.

The invention relates to novel fast-disintegrating, or even instant-disintegrating, homogeneous microporous compositions for pharmaceutical, veterinary, food, dietetic or cosmetic use, intended for the oral route or to be applied in contact with the mucous membranes and a method for producing them.

Fast-disintegrating or instant-disintegrating solid compositions for the oral route have for a very long time been of interest to formulators and also to practitioners and patients who find in them interesting characteristics in terms of compliance. As regards very young or old subjects in whom deglutition of solid forms poses problems, the compositions as provided in the present invention offer a real advantage because they can be taken either in a glass of water or directly under the tongue where they disintegrate instantly.

By virtue of these characteristics, the compositions which are the subject of the invention represent the ideal solution for an ambulatory treatment.

Furthermore, they respond favorably to the unconscious association made by the patient between speed of dissolution or of disintegration of the composition and speed of action of the molecule, especially for analgesics, antinauseants, antiulceratives, anti-asthmatics and antianginals. This unconscious association being sometimes able to enhance the efficacy of the molecule.

The expression fast-disintegrating form is understood to mean galenic forms whose disintegration remains less than 15 minutes in accordance with the tablets monograph (Compressi) of the French or European pharmacopoeia.

Several fast-disintegrating formulations are already used in the pharmaceutical field. Effervescent tablets or granules allow disintegration in less than 5 minutes through the fast dissolution or dispersion of the molecule by virtue of the controlled release of carbon dioxide gas obtained from an acid-base chemical reaction.

This technology, which is currently very widely used and is described in many patents (EP 673 644; EP 369 228; FR 2 552 308), remains mastered at the industrial level by few companies. Indeed, this technique requires a substantial know-how in the carrying out of the wet granulation step, but also a controlled humidity environment which is very expensive to maintain.

Furthermore, the substantial size and effervescence of the form do not make it possible to use conventional effervescent tablets in the buccal cavity or in the absence of water.

This problem has been solved in novel formulations called microeffervescent formulations which were the subject of the recent American patent U.S. Pat. No. 5,178,878.

Water-dispersible tablets or granules constitute fast-disintegrating forms whose property is essentially based on the use of compounds called superdisintegrants. Upon contact with water, they produce, through their very high swelling power, “the explosion” of the compressed or granular mass.

Many patents describe this type of galenic forms (FR 95/00947, EP 0 347 767, EP 0 716 852 and EP 0 361 354) and the great majority uses the following compounds: starch glycolate, microcrystalline cellulose, carboxymethyl cellulose and polyvinylpyrrolidone which are crosslinked.

Some authors use less common disintegrants such as clays of the smectite or actapulgite type (WO 92/13527), or gums and more particularly guar gum (EP 0 273 005).

As for the effervescent tablets, these forms are very difficult to use without water and therefore poorly suited to ambulatory buccal or sublingual use. It is also necessary in very many cases, to increase the volume and thus the weight of the tablet in order to have a specific surface area compatible with fast disintegration.

The formulation of this type of tablet which may appear to be simple at first glance, is in fact quite complex and is based on a compromise between hardness and disintegration which has to be optimized as much as possible, according to the physico-chemical nature and the amount of active ingredient.

Recently, patent EP 764 019 describes the development, using sugars amorphized by extrusion, of fast-disintegrating forms by a method minimizing the compression phase (compaction with compressing-metering device). Given the low hardness of the compacts, the company holding this novel form had to solve the packaging (blister type) step by adapting methods which are not very compatible with industrial throughputs.

Furthermore, the effervescent and water-dispersible tablet technologies are based on batch processes including a phase of compressing one or more pulverulent mixtures.

This necessarily results in a low production throughput compared with a continuous process and, consequently, an increase in the production cost.

In parallel with the preceding two tablet forms, solid unit forms exist in the pharmaceutical field which are manufactured by lyophilization, called oral lyophilizates.

This lyophilization technology has been known for years (FR 2 403 078) and is used to preserve and administer molecules which are sensitive from the physico-chemical point of view.

This clumsy and expensive technology, in which the duration of lyophilization at the industrial level is close to 24 hours, whose energy consumption is high (5 kW/h per kg of water), does not allow, in contrast to the present invention, application, for economic reasons, to all products.

However, through the use of judiciously chosen excipients, the lyophilization makes it possible to obtain forms exhibiting fast-disintegration either in contact with a suitable volume of water or after bringing into contact with saliva.

Many recent documents describe this type of galenic forms (GB 2 111 423, U.S. Pat. No. 5,039,540, U.S. Pat. No. 5,120,549, WO 94 14422 and EP 651 997, EP 399 902).

Advantageously, these lyophilizates are suitable for ambulatory buccal and sublingual use. On the other hand, during the bringing into contact with the buccal mucous membrane, the solid powders used in the formulation confer an unpleasant, distinctly perceptible granular sensation. Furthermore, regardless of the fast-disintegrating forms used, their delicate and not very flexible mode of preparation does not make it possible to adapt the rate of disintegration according to the use requirement.

The object of the present invention is to provide novel compositions and their method of production as described below and illustrated in the examples, which make it possible to obtain disintegration times which are equal to or even less than oral lyophilizates. Like the latter, the novel form may be dissolved, either with a suitable volume of water, or directly in the mouth or in contact with the mucous membranes.

On the other hand, the compositions according to the invention, by virtue of their formulation and their continuous method of production comprising a phase of mixing the components, of extruding or injecting the pasty composition into a blister, and then a continuous microwave drying-forming phase under vacuum, have a completely different texture where the solid particles solubilized at one moment of the process are no longer perceptible during the bringing into contact with the buccal mucous membrane. Furthermore, the continuous method of production at the pilot or industrial level allows, through its adaptability (time as a function of the volume) and its lower energy consumption, it to be a lot less expensive than the lyophilization method.

The composition according to the invention for pharmaceutical, veterinary, food, dietetic or cosmetic use and affording fast dissolution in an aqueous medium or on contact with the mucous membranes comprises 1% to 50% by weight of one or more active ingredient(s), 50% to 99% by weight of a carrier comprising one or more polymer(s), optionally one or more diluent(s) and optionally one or more additive(s), in particular a flavoring or a coloring, said composition being characterized in that it has a fast-dissolving isotropic microporous expanded structure and the polymers being chosen from the group consisting of polymers of plant origin, optionally in combination with polymers of animal origin or synthetic polymers, and said carrier being such that the binding polymer(s) is/are present in the composition in a proportion greater than or equal to 1% (w/w) and more particularly of between 6% and 98% (w/w).

The composition has a porous structure, especially a density of less than 0.9 g/cm³.

The composition has also a compressive strength superior or equal to 30N, advantageously to 35N.

It has also a stress at break superior or equal to 3.10⁵ Pa, advantageously superior or equal to 3.5.10⁵ Pa.

The composition has a cleavable structure. In particular, the composition can be divided in at least two parts (or more) of equal dimension by hand without any problem. Therefore, this composition is not too brittle and is less brittle than freeze-dried products.

Furthermore, the composition according to the present invention can be handled by hand without any problem contrary to freeze-dried products which are too brittle.

Moreover, because of its compressive strength, the composition according to the present invention can be expelled from the blister without the drawbacks of the freeze-dried products, i.e. without crumbling away. Therefore, the membrane seal of the blister do not need to be a peelable film as this is the case for freeze-dried products but can be any type of films such as aluminium foil or heat-sealable films.

A disintegration test which is appropriate because it illustrates the behavior during disintegration of the compositions consists in placing the composition in a beaker containing 100 ml of water whose temperature is between 15 and 25° C. The time necessary for the entire form to be dissolved is noted.

On the other hand, the USPXXIII apparatus No. 2 method termed paddle apparatus using, as dissolution medium, distilled water at 37° C. and a paddle rotating speed of 50 RPM was used as in vitro dissolution test.

In the case of the so-called expanded form, the expansion level refers to the ratio of the volume of the compositions after drying-forming to the ratio of the volume before drying.

This change in volume also being accompanied by a variation in the density.

This novel pharmaceutical, veterinary, dietetic, food or cosmetic form in which the homogeneous and controlled expansion of the polymer by virtue of the operating conditions of the microwave drying-forming phase under vacuum makes it possible to obtain an isotropic porous structure then conferring a rate of disintegration in water or the buccal cavity or on contact with the mucous membranes which may range from a few seconds to several minutes depending on the use requirement.

The novelty of this invention is also based on the choice of the polymer(s), of the diluent(s) used for the constitution of the matrix network of the form, but also on the method of production which makes it possible to continuously produce, in a time of less than 1 hour, preferably of less than 30 minutes, forms whose porosity and form can be modulated during the continuous microwave drying-forming phase under vacuum.

Among the active ingredients which are suitable for producing the composition according to the invention, there may be mentioned as a guide and without limitation the active ingredients chosen from the group consisting of medicaments and food additives.

The active ingredients used have a very different solubility such as Milnacipran (aqueous solubility equal to 800 g/l), piroxicam and domperidone (aqueous solubility of less than 100 mg/l) and phloroglucinol (aqueous solubility in the region of 30 g/l).

There may also be mentioned, without limitation, as antimigraine analgesics, derivatives of ergot of rye (ergotamine, dihydroergotamine, methysergide) or serotonin antagonists (cyproheptadine, pizotifen, oxeterone). As antipyretic analgesics and/or anti-inflammatory agents derived from arylcarboxylics, there may be mentioned salicylic acid, acetylsalicylic acid, mefenamique acid. As antipyretic analgesics and/or anti-inflammatory agents derived from arylalkanoic acids, there may be mentioned diclofenac, indometacin and as antipyretic analgesics and/or anti-inflammatory derivatives of enolic acids, there may be mentioned phenylbutazone and tenoxicam. As local anesthetics, there may be mentioned lidocaine and tetracaine. As antianginals, there may be mentioned isosorbide 5-mononitrate, molsidomine. As anticholinergic antispasmodics, there may be mentioned metoclopramide, loperamide, mebeverine, papaverine, trimebutine. As antisecretory agents, there may be mentioned cimetidine, ranitidine. As muscle relaxants, there may be mentioned diazepam, progabide, dantrolene, mephenesin, baclofenen, antiulceratives (in the broad sense), antihypertensives, conversion enzyme inhibitors, angiotensin II antagonists, antagonists of calcium β-blockers, central peripheral vasodilators, coronary vasodilators, antiarrhythmics, platelet aggregation inhibitors, antibiotics, oral corticoids, antimigraines, antipsychotics, hypnotics, sedatives and antinauseants.

The polymer according to the invention should satisfy two conditions which are often contradictory, namely, on the one hand, its binding character allowing it to be extruded or injected and then formed and, on the other hand, its instant disintegrating capacity after having been subjected to the drying-forming method.

The physico-chemical properties, the particular concentration which is not very high for fast-disintegrating forms of the matrix polymer(s) and the drying-forming conditions are important criteria because they strongly influence the porosity and the forming by expansion of the form and therefore the rate of disintegration, therefore imposing a rigorous choice of these polymers from the point of view of the chemical structure and the molecular mass, but also a precise control of the vacuum and heat energy parameters used for the implementation of the invention.

Indeed, certain polymers, by virtue of their excessively pronounced hydrophobic character, will not be suitable because whatever their molecular mass, they cannot be dispersed and formulated in an aqueous medium in a viscosity range allowing their distribution by injection or extrusion. Other hydrophilic polymers with excessively high molecular weight or too sensitive to a rise in temperature do not make it possible to achieve the objective of the invention either.

By contrast, poor control of the operating conditions for drying-forming (vacuum, heat energy, duration) leads, according to the formulation, to forms which are non porous or of heterogeneous porosity or have excessively expanded structures incompatible with the use according to the invention.

These criteria will vary according to the type of polymers or the combination of polymers chosen.

However, it has been observed, in general, that the hydrophilic polymer ought to be in an interval of average molecular mass of between 1000 and 2,000,000 Da, given that for each polymer, a sub-interval of molecular mass can be easily determined by persons skilled in the art, in particular by the disintegration tests indicated above.

Among these polymers, there may be mentioned in particular polysaccharides of plant origin obtained by chemical or enzymatic hydrolysis from native starches. Among the polysaccharides of plant origin obtained by chemical or enzymatic hydrolysis from native starch, there may be mentioned in particular those which correspond with the definition of maltodextrin or of glucose syrup. Preferably, the polymer of plant origin of the polysaccharide type obtained by chemical or enzymatic hydrolysis is chosen from maltodextrins or glucose syrups having dextrose equivalent (DE) levels of between 3 and 50 and preferably between 6 and 34 or mixtures thereof.

There may also be mentioned chemically modified polysaccharides of plant origin. The expression chemically modified starch is understood to mean sodium glycolate of starch. Among the hydrophilic polymers, there may also be mentioned chemically modified polymers derived from cellulose, alkyl celluloses such as hydroxypropyl cellulose (HPC), hydroxypropyl methyl cellulose (HPMC), hydroxyethyl cellulose, low or medium viscosity carboxymethyl cellulose sodium (CMCNa).

There may also be mentioned polymers of the gum type. As a polymer of the gum type, there may be mentioned guar, gum arabic, xanthane gum, pectin and alginates or mixtures thereof.

Among the synthetic polymers, there may be mentioned polyethylene glycols (PEG), polyvinylpyrrolidone (PVP).

Among the polymers of animal origin, there may be mentioned proteins such as gelatin, collagen, sodium caseinates, chondroitic acid sulfate and hydrolysates thereof, chitosans and soluble hydrolysis derivatives thereof or mixtures thereof.

The mixtures of these various polymers in appropriate proportions are also envisaged. Indeed, for example in the case of a maltodextrin/PVP mixture, there is formation of very soluble microporous structures.

Preferably, the said polymer(s) is/are present in the formulation at a percentage compatible with a viscosity of between 100 mPa·s and 100,000 mPa·s, preferably between 100 and 50,000 mPa·s.

Among the diluents, there may be mentioned mannitol, sucrose, lactose, fructose, sorbitol, xylitol, maltitol and dicalcium phosphate dihydrate.

The composition according to the invention may comprise up to 10% of additives. These additives are in particular chosen from the group consisting of plasticizers, flavorings, colorings, opacifiers.

Preferably, the composition for pharmaceutical or food use according to the invention has a disintegration time of between 1 second and 10 minutes, preferably of less than 1 minute, advantageously of less than 30 seconds, when taken by the patient whether in the presence of an appropriate volume of water or on direct contact with the buccal mucous membrane or any other mucous membrane to which the microporous expanded form is applied.

It is also possible, according to an advantageous variant, to characterize the composition by its density, preferably of between 0.1 and 0.9 g/cm³, advantageously between 0.2 and 0.7 g/cm³.

In addition, the composition according to the invention is such that the active ingredient(s) in the expanded microporous or porous matrix is/are in the dissolved or dispersed state or in film-coated forms.

According to an advantageous embodiment, the final packaging is polypropylene or polytetrafluoroethylene (Teflon®).

The invention also relates to a method for preparing the compositions according to the invention comprising the mixing of the active ingredient, diluents and polymers and additives followed by extrusion or direct injection into a mould or blister according to the viscosity of the formulation, this mould or blister and the drying method make it possible to give the composition its final form.

This so-called compact composition is subjected to an instant microwave continuous dielectric treatment under vacuum, optimally bringing about at the same time the drying of the form, the creation of porosity and the forming while avoiding reaching excessively high heat levels which can induce degradation of the active ingredient.

The composition is then recovered and packaged, preferably in the context of a continuous process.

Optionally, the composition present in the mould or blister is recovered before the drying step, advantageously by an aluminium foil (for example with a thickness of 20 to 30 μm) or a vapor semipermeable complex. In this case, advantageously, the blister is made in prolypropylene, more advantageously with a thickness of between 200 to 400 μm.

According to a general method of use, the method for preparing a fast-disintegrating composition for pharmaceutical, veterinary, food, dietetic or cosmetic use according to the invention is characterized in that a pasty formulation comprising one or more active ingredients, one or more polymers, one or more diluents and optionally one or more additives is homogenized, it is injected into a blister, and then in that the form is dried-expanded and molded by a microwave-type method under vacuum, to give rise to an isotropic expanded microporous structure, in particular having a density of less than 0.9 g/cm³.

Preferably, the method for preparing a fast-disintegrating composition for pharmaceutical or food use is characterized in that the drying-forming and control of the porosity are carried out during a simultaneous operation and is such that the vacuum level used is between 30 to 700×10² Pa and preferably between 60 and 500×10² Pa (30 to 700 mbar and preferably between 60 and 500 mbar) to give rise to an isotropic expanded microporous structure of regular form, in particular having a density of less than 0.9 g/cm³.

Advantageously, the method for preparing a fast-disintegrating microporous composition for pharmaceutical, veterinary, food, dietetic or cosmetic use is characterized in that the pasty formulation obtained by homogenization has a viscosity of between 100 mPa·s and 100,000 mPa·s, preferably between 100 and 50,000 mPa·s, followed by injection or extrusion of this mass into a blister which may be advantageously the final packaging. Preferably, the temperatures during the drying and forming phase are between 25° C. and 80° C., thereby avoiding the degradation of the heat-labile active ingredients.

The duration of the drying and forming operation is advantageously less than 1 hour, preferably 30 minutes.

According to an advantageous variation, the blister is the final packaging having a chemical nature of polypropylene or polytetrafluoroethylene type.

### BRIEF DESCRIPTION OF THE DRAWINGS

FIG. 1 shows a photograph (magnification factor 4) of the microporous form described in Operating condition a.

FIG. 2 shows a photograph (magnification factor 4) of the microporous expanded form described in Operating condition b.

FIG. 3 shows a photograph (magnification factor 5.5) of the microporous expanded form described in Example 2a.

FIG. 4 shows a photograph (magnification factor 4) of the expanded porous structure described in Example 6b.

The invention will now be illustrated without limitation by the following examples:

### EXAMPLE NO. 1

A mixture (MD1) composed of 40% of water, 56% of Maltodextrin having a DE in the region of 19 and 4% of orange flavor whose viscosity is in the region of 600 mPa·s is distributed (about 0.7 to 1 ml) into polypropylene blisters.

These samples are introduced one after the other into a microwave oven, connected to a vacuum pump, and subjected to various operating conditions.

The process is thus carried out and monitored continuously by controlling the energy levels applied to the sample, the temperature of the product and the level of vacuum applied to the sample.

Operating Condition a:

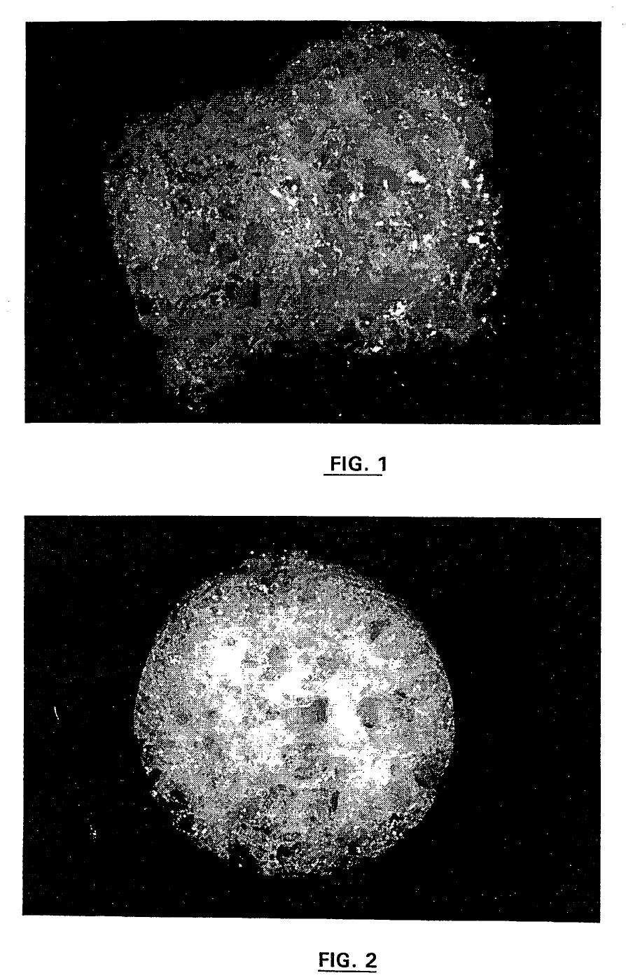

The sample is injected into its polypropylene blister and then subjected to a vacuum level of 20×10² Pa (20 mbar) and a microwave power such that the sample absorbs about 11 W during the 10 minutes of the process. Under these experimental conditions (1a), the sample very rapidly undergoes uncontrolled expansion and drying, leading to a non isotropic expanded microporous form described as buffed as illustrated on the photograph of FIG. 1 with a magnification factor of 4, incompatible with a use in the pharmaceutical or food sector.

Operating Condition b:

Another sample (0.7 ml) is injected into its polypropylene blister and is subjected for 15 minutes to microwaves with a pressure level of 60×10² Pa (60 mbar).

Under these experimental conditions (1b), the sample absorbs between 3 and 4 W and undergoes a controlled expansion and drying, leading to an isotropic microporous expanded form having a density in the region of 0.22 and a volume in the region of 3 cm³, in agreement with the objective in relation to morphology and disintegration. An example of the forms obtained under these conditions on the photograph in FIG. 2 (magnification factor 4).

Indeed, the samples manufactured according to these experimental conditions exhibit disintegrations of 30 seconds in a glass of water and of the order of about ten seconds in the mouth.

Operating Condition c:

Another sample (1c) is injected into its polypropylene blister and is subjected for 20 minutes to an exposure power such that it absorbs 2.5 W and a vacuum level of 90×10² Pa (90 mbar).

Under these experimental conditions (1c), the sample undergoes controlled expansion and drying, leading to an isotropic microporous expanded form having a density in the region of 0.22 in agreement with the objective in terms of morphology and disintegration.

Indeed, the samples manufactured according to these experimental conditions exhibit disintegrations of 30 seconds in a glass of water and of the order of about ten seconds in the mouth.

Operating Condition d:

Another sample (1d) is injected into its polypropylene blister and is subjected for 15 minutes to an exposure power such that it absorbs about 3.5 W and a vacuum level of 90×10² Pa (90 mbar) for 5 minutes and then 60×10² Pa (60 mbar) for 10 minutes.

Under these experimental conditions (1d), the sample undergoes a controlled expansion and drying, leading to an isotropic microporous expanded form having a density in the region of 0.2 in agreement with the objective in terms of the morphology and the disintegration.

Indeed, the samples manufactured according to these experimental conditions exhibit disintegrations of 35 seconds in 100 ml of water and of the order of about ten seconds in the mouth.

This example perfectly illustrates the invention from the point of view of its process in the sense that the same basic formula, subjected to various microwave drying conditions under vacuum leads to fast-dissolving isotropic microporous expanded forms having completely different and controllable porosity and size uniformity.

Indeed, the drying method according to the invention surprisingly allows, through a judicious choice and monitoring of the operating conditions, product temperature and vacuum level, to manage the drying, the creation of porosity and the forming of the finished product.

In the examples presented, the source of dielectric energy is the microwave but for considerations of compatibility (degradability, dielectric reactivity) with the formulation or industrial necessities (speed of the process or technological choices), this mode of energy supply may be optionally and advantageously replaced by high frequencies.

### EXAMPLES NO. 2

Example 2a: An isotropic microporous expanded form containing 490 mg of maltodextrin (DE 19), 10 mg of orange flavoring and 100 mg of phloroglucinol dihydrate is obtained after having subjected a pasty mixture having a viscosity in the region of 3000 mPa·s to the experimental conditions previously described in (1b).

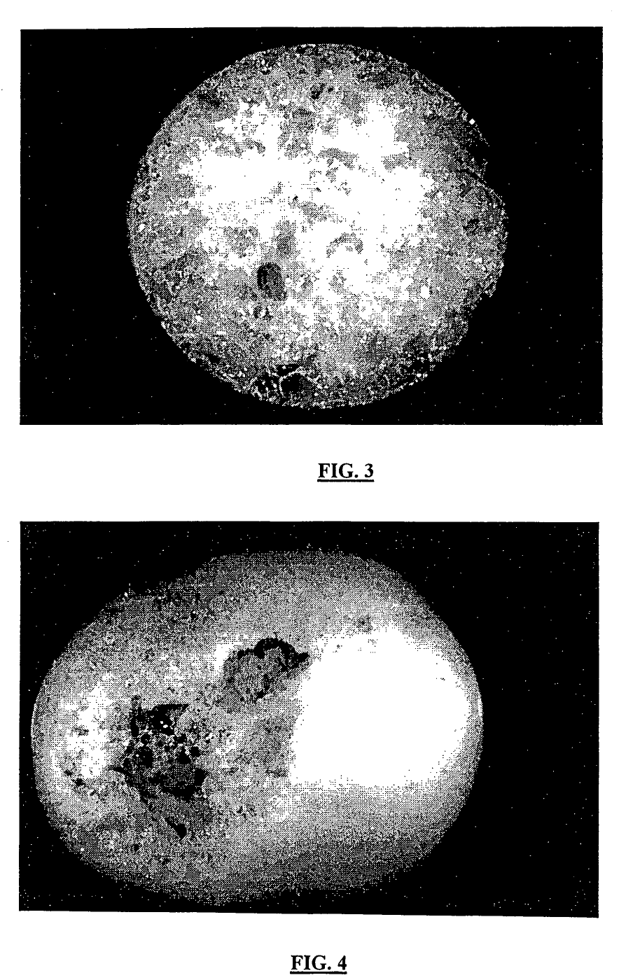

The isotropic microporous expanded form obtained having a density in the region of 0.21 and a volume of 2.80 cm³ exhibits characteristics of disintegration and form in agreement with the objectives (32 seconds) as illustrated in FIG. 3 (photograph with magnification factor of 5.5).

Example 2b: A form having the same composition but having an uncontrolled expansion level as well as a very heterogeneous microporous expanded structure is obtained by subjecting the same mixture to pressure conditions of 30×10² Pa (30 mbar) and an absorbed power of 4 W. This form, although in agreement with the disintegration objective (about 30 seconds) is not in agreement with the form objectives given the irregularity of the surface and of the internal network obtained.

### EXAMPLE NO. 3

An isotropic microporous expanded form containing 588 mg of maltodextrin (DE 19), 10 mg of mint flavor and 100 mg of phloroglucinol is obtained by subjecting a mixture having a viscosity in the region of 3000 mPa·s to the conditions previously described (1b).

The isotropic microporous expanded form has a controlled expansion level (final volume of 2.75 cm³) a density in the region of 0.21 and disintegrates within 30 seconds in 100 ml of water and of the order of about ten seconds in the mouth.

### EXAMPLE NO. 4

An isotropic microporous expanded form containing 572 mg of maltodextrin (DE 19), 10 mg of mint flavor, 10 mg of xylitol and 100 mg of phloroglucinol is obtained by subjecting a mixture having a viscosity in the region of 3100 mPa·s to the conditions previously described (1b).

The isotropic microporous expanded form obtained in agreement with the objectives has an expansion level (final volume of 2.95 cm³) a density in the region of 0.22 and disintegrates within about 32 seconds in 100 ml of water and practically instantly in the mouth.

### EXAMPLE NO. 5

An isotropic microporous expanded form containing 455 mg of maltodextrin (DE 19), 102 mg of PVP, Kollidon 12 PF type, 20 mg of natural mint flavor, 20 mg of xylitol and 100 mg of phloroglucinol is obtained by subjecting a mixture having a viscosity in the region of 3000 mPa·s to the conditions previously described (1b).

The form obtained in agreement with the objectives has an expansion level (final volume of 2-75 cm³ a density in the region of 0.2, disintegrates within about 30 s in 100 ml of water and instantly on contact with the buccal mucous membrane.

### EXAMPLES 6

Example 6a: An isotropic microporous expanded form having the following composition 515 mg of Maltodextrin (DE 19) and 85 mg of milnacipran is obtained after having subjected to the process a mixture having a viscosity in the region of 2800 mPa·s under the conditions described in example 1b.

This isotropic microporous expanded form has a density in the region of 0.25 and disintegrates in 30 seconds in 100 ml of water and instantly on contact with the buccal mucous membrane.

Example 6b: A mixture of the same composition subjected to the same conditions of energy power but to lower pressure levels of the order of 40×10² Pa (40 mbar) has an expanded porous structure of uncontrolled form and size as illustrated in the photograph of FIG. 4 with a magnification of 4 not compatible with a use in the pharmaceutical field.

### EXAMPLES 7

Example 7a: An isotropic microporous expanded pharmaceutical form having the composition 515 mg of maltodextrin (DE 19), 85 mg of piroxicam is obtained, after having introduced into a polypropylene blister a mixture having a viscosity in the region of 3500 mPa·s. This mixture is subjected in a microwave under vacuum to the following conditions: 3.3 W absorbed by sample and a vacuum level of 70×10² Pa (70 mbar) for 10 minutes.

Under these experimental conditions (7a), the samples have a structure in accordance with the objective with an expansion level in the region of 3.5 and a disintegration of 35 seconds in 100 ml of water and instantly in contact with the buccal mucous membrane.

Example 7b: Under different experimental conditions, namely 8 W absorbed by sample and a vacuum level of 30×10² Pa (30 mbar) for 7 minutes, the form obtained having the same composition although in accordance with the objectives in terms of disintegration is not suitable in terms of form.

### EXAMPLE NO. 8

An isotropic microporous expanded pharmaceutical form having the composition 515 mg of maltodextrin (DE 19) and 85 mg of domperidone in agreement with the objectives according to the invention is obtained, after having introduced into a polypropylene blister a mixture having a viscosity in the region of 3500 mPa·s. This mixture is subjected in the microwave oven under vacuum to the following conditions: 3 W absorbed by sample and a vacuum level of 65×10² Pa (65 mbar) for 10 min.

### EXAMPLE NO. 9

An isotropic microporous expanded pharmaceutical form having the composition 100 mg of maltodextrine (DE 19), 650 mg of mannitol and 50 mg of piroxicam is obtained after having subjected to the drying process (between 90×10² and 500×10² Pa (90 and 500 mbar) for 0.5 h) a pasty composition having a viscosity of 2000 mPa·s. Under these judiciously chosen operating conditions, the form obtained has morphological characteristics of disintegration in agreement with the objectives.

### EXAMPLE NO. 10

Under experimental conditions described in example 1b, it was possible to obtain instant-disintegrating isotropic microporous expanded pharmaceutical forms having the composition 100 mg of phloroglucinol, 40 mg of sodium caseinate, 20 mg of xylitol and 400 mg of mannitol.

### EXAMPLE NO. 11

In a similar manner, pharmaceutical forms of the following composition, namely 100 mg of phloroglucinol, 50 mg of chitosan and 400 mg of maltodextrin having a DE in the region of 19 were able to be obtained. These forms have morphological and disintegration characteristics in agreement with the objectives.

### EXAMPLE NO. 12

Mixtures based solely on maltodextrin or glucose syrup having different dextrose equivalents (6, 14, 21, 34) flavored either with orange or mint flavor or with coffee extract and initially containing 30 to 40% of water, made it possible, after having been subjected to microwaves under vacuum (90×10² to 500×10² Pa (90 to 500 mbar) for 0.5 h) the obtaining of expanded microporous forms instantly soluble in water and in agreement with the objective in terms of the form. These isotropic microporous expanded single-dose compositions may be easily used as refreshing drinks.

### EXAMPLE 13

Isotropic microporous expanded forms containing 500 mg of lactose, 40 mg of Maltodextrin (DE 19) and 50 mg of piroxicam were obtained by subjecting a mixture having an initial water content of the order of 20% (w/w) to modulation of the experimental conditions, by reducing in particular the microwave power transmitted to the sample and by working at pressure values of between 100×10² and 500×10² (100 and 500 mbar) for 0.5 h.

These forms have, after exposure to the treatment of the invention, a water content of less than 1% of the total mass.

These isotropic microporous expanded forms have a disintegration time in agreement with the objective.

### EXAMPLE 14

Isotropic microporous expanded forms containing 500 mg of lactose, 30 mg of carboxymethyl cellulose sodium (low viscosity) and 10 mg of piroxicam were obtained by subjecting to the experimental conditions 13 a mixture having an initial water content of the order of 30% (w/w).

These forms have, after exposure to the treatment of the invention, a water content of less than 1% of the total mass.

These isotropic microporous expanded forms have a disintegration time in agreement with the objective.

### EXAMPLE 15

Isotropic microporous expanded forms containing 500 mg of lactose, 10 mg of xanthan gum+60 mg of maltodextrin of DE 34 and 10 mg of piroxicam were obtained by subjecting to the experimental conditions 13 a mixture having an initial water content of the order of 30% (w/w).

These forms have, after exposure to the treatment of the invention, a water content of less than 1% of the total mass.

These microporous forms have a disintegration time in agreement with the objective.

### EXAMPLE 16

A batch of 500 microporous expanded forms containing 450 mg of mannitol, 67 mg of maltodextrin of DE 19, 7 mg of mint flavor and 21 mg of piroxicam was obtained in 30 min on an industrial microwave tool under vacuum under conditions similar to the operating conditions previously described in example 13.

The forms obtained having morphological and disintegration characteristics in agreement with our objectives proved, in addition, stable after having been subjected to an accelerated stability study at 40° C./75% Relative Humidity for 6 months.

### EXAMPLE 17

A batch of 500 microporous expanded forms containing 450 mg of mannitol, 67 mg of maltodextrin of DE 19, 7 mg of mint flavor and 21 mg of domperidone was obtained in 30 minutes on an industrial microwave tool under vacuum under conditions similar to the operating conditions previously described in example 13.

The forms obtained having morphological and disintegration characteristics in agreement with our objectives proved, in addition, stable after having been subjected to an accelerated stability study at 40° C./75% Relative Humidity for 6 months.

### EXAMPLE 18

### Comparative Textural Properties of Freeze-Dried Product/Product According to the Present Invention Obtained by Means of Microwave Vacuum

The forms tested (Freeze-dried product and product according to the present invention obtained with microwave vacuum) correspond to the same formula:

| | |
|---|---|
| Maltodextrin | 60.57 mg |
| D mannitol | 434.23 mg |
| Strong Mint flavour | 5.16 mg |
| Polysorbate 80 | traces |

1. Characterisation Evaluation

| | | Product |
|---|---|---|
| | | according |
| | Freeze- | to the |
| | dried | present |
| | tablet | invention |
| Water content | 0.025 | 0.011 |
| Expansion rate | 0.91 | 1 |
| Porosity | 0.356 | 0.359 |
| Disintegration | 181.5 | 78.25 |
| time (s) | | |
| Stress at | 2.62 | 3.91 |
| break (Pa) ×10⁵ | | |
| density | | 0.655 |

Conclusion:

A microwave vacuum-dried sample according to the present invention is expanded more than a freeze-dried tablet but is more resistant to crushing (50% increase) than a freeze-dried tablet with shorter disintegration times.

2. Devices Used and Related Results

2.1. Disintegration Time

The disintegration times of the pharmaceutical forms are measured using the experimental devices below.

The main components are:
a stainless steel container in which the base is a 710 μm calibrated screen
a graduated metal weight placed on the sample
a beaker containing a 4 cm long bar magnet
a water inlet at the base of the beaker

The measurement of the sample disintegration or dissolution time, for 100 ml of purified water poured into the device and under stirring at 300 rpm, is equivalent to the time taken by the metal weight to move from its initial position (with sample) to its final reference position (contact between screens) with an error of ±7 s.

Measurements and Results

TABLE 1
Disintegration time data evaluation on samples with the same
formulation obtained by means of freeze-drying
and microwave vacuum

| | | | Product |
|---|---|---|---|
| | | | according to |
| | | | the present |
| | | Freeze- | invention |
| | Sample | dried | (Microwave |
| | No. | tablet | vacuum) |
| Disintegration | 10C | 218 | 77 |
| time | 10D | 145 | 80 |
| | 1A | | 52 |
| | 1B | | 104 |
| | 9B | | |
| | mean | 181.5 | 78.25 |
| Water content | | 0.025 | 0.011 |

2.2. Crushing Resistance or Measurement of Stress at Break by Brazilian Tests

The Brazilian test is a standardised test (ISRM, 1978) used to determine the stress at break of different materials. It consists of subjecting a cylindrical sample to radial compression which results in tensile stress being applied to the material at the centre of the sample.

Assuming an isotropic linear elastic behaviour of the material, the tensile stress developed at the centre of the sample (Equation 1) can be deducted from the force (F) applied and the dimensions of the cylinder (mean diameter d and length L) with an error generally estimated at approximately 20%.

$$σ = \frac{2 F}{π d L} \qquad Equation 1$$

The stress at break is then obtained for the force Fmax inducing a crack perpendicular to the compression platen.

During an experiment, the crushing speed is fixed, a force sensor is used to monitor the variation in the load over time. The result of an experiment can be represented as the variation of the tensile stress at the centre of the sample as a function of its deformation. The stress at break corresponds to the time at which there is a sudden drop in the mechanical stress, indicating a collapse of the structure and the splitting of the sample into two parts.

TABLE 2
Crushing resistance data evaluation on samples
with the same formulation obtained by means of freeze-
drying and microwave vacuum

| | | Product |
|---|---|---|
| | | according |
| | | to the |
| | | present |
| | Freeze- | invention |
| | dried | (Microwave |
| | tablet | vacuum) |
| Fmax Mean | 24.6 | 39.8 |
| Deformation | 0.998 | 1.102 |
| (mm) Mean | | |
| Stress σₘₐₓ | 2.62 E+05 | 3.91E+05 |
| (Pa) Mean | | |

2.3. Porosity and Pore Size Distribution Study

Mercury porosimetry is used to measure the pore access radii. These radii may vary between 18 Angströms and 70 microns. The experiment is based on the physical principle that a non-reactive, non-wetting liquid (in this case, mercury) can only penetrate into the pores if a pressure consistent with the pore radius is applied. The relationship between the pressure required and the size of the pores in which the mercury can penetrate is given by the Washburn equation:

$$P = \frac{2 γ cos θ}{r} \qquad Equation 2$$

Where P is the pressure applied;
r is the pore access radius;
y the interfacial tension between the mercury and the sample (480 dyne/cm);
θ the wetting angle between the mercury and the pore wall (generally approximately 140° as mercury is non-wetting):
   In practice, we obtain the distribution of the volume introduced into the pores as a function of the pressure applied and therefore the pore access radii.

Pore sizes can be classified into different groups: micropores (<10 Å), mesopores (between 10 and 100 Å), macropores (between 100 and 37 500 Å), ultramacropores (>37 500 Å).

Measurements and Results

The presence of pores with a radius between 20 and 50 microns only occurs on the forms obtained by microwave vacuum drying, which explains the shorter disintegration times (the liquids penetrate more easily inside the form).

In conclusion, the form obtained by microwave vacuum drying displays, for the same porous volume, a different pore distribution, a higher crushing resistance and a shorter disintegration time with respect to a freeze-dried product with the same formula.

1. A fast dissolving composition for pharmaceutical, veterinary, food, dietetic, or cosmetic use, comprising 1% to 50% by weight of one or more active ingredients and 50% to 99% by weight of a carrier comprising one or more polymers, wherein the composition has a fast-dissolving isotropic microporous expanded cleavable structure, a density of less than 0.9 g/cm³ and a compressive strenght equal or superior to 30N and wherein the polymers are chosen from the group consisting of polymers of plant origin, optionally in combination with polymers of animal origin or synthetic polymers, the polymers being present in the composition in a proportion greater than or equal to 1% (w/w).

2. The composition according to claim 1 wherein the polymers are present in a proportion of between 6 and 98% (w/w).

3. The composition of claim 1, wherein the polymer of plant origin is selected from polysaccharides obtained by chemical or enzymatic hydrolysis of chemically modified starch, polymers of a chemically modified cellulosic type, and polymers of a gum type, or mixtures thereof.

4. The composition of claim 3, wherein the polysaccharide is selected from maltodextrins or glucose syrups, and sodium glycolates of starch and mixtures thereof.

5. The composition of claim 4, wherein the polymer of plant origin is selected from maltodextrins and glucose syrups having a dextrose equivalent (DE) level of between 3 and 50, and mixtures thereof.

6. The composition of claim 5 wherein the dextrose equivalent level is of between 6 and 34.

7. The composition of claim 3, wherein the polymer of plant origin of the cellulosic type is selected from carboxymethyl cellulose sodium of low or medium viscosity, hydroxypropyl methyl cellulose, hydroxypropyl cellulose, hydroxyethyl cellulose and mixtures thereof.

8. The composition of claim 3, wherein the polymer of plant origin is of the guar gum, gum arabic, xanthane, pectin and alginate type, or mixtures thereof.

9. The composition of claim 1, wherein the synthetic polymer is polyvinylpyrrolidone.

10. The composition of claim 1, wherein the polymer of animal origin is selected from sodium caseinates, chitosan, their water-soluble hydrolysis derivatives, gelatin, collagen, chondroitic acid sulfate, hydrolysates thereof, and mixtures thereof.

11. The composition of claim 1, wherein the polymer(s) is/are present in the formulation at a percentage at least equal to 1% (w/w) and compatible with a viscosity of between 100 mPa·s and 100,000 mPa·s.

12. The composition of claim 2, wherein the polymer(s) is/are present in the formulation at a percentage of between 6 and 98% (w/w) and compatible with a viscosity of between 100 mPa·s and 100,000 mPa·s.

13. The composition of claim 11, wherein the polymer(s) are present in the formulation at a percentage at least equal to 1% (w/w), and compatible with a viscosity of between 100 mPa·s and 50,000 mPa·s.

14. The composition of claim 12, wherein the polymer(s) are present in the formulation at a percentage of between 6 and 98% (w/w), and compatible with a viscosity of between 100 mPa·s and 50,000 mPa·s.

15. The composition of claim 1, wherein the composition comprises a diluent selected from mannitol, sucrose, lactose, fructose, sorbitol, xylitol, maltitol and dicalcium phosphate dihydrate.

16. The composition of claim 1, wherein the density is between 0.2 and 0.7 g/cm³.

17. The composition of claim 1, wherein the composition has a disintegration time of less than 1 minute under conditions of use on direct contact with a mucous membrane, in particular the buccal mucous membrane, or in an appropriate volume of water.

18. The composition of claim 1, wherein the composition has a disintegration time of less than 30 seconds under conditions of use on direct contact with a mucous membrane, in particular the buccal mucous membrane, or in an appropriate volume of water.

19. The composition of claim 1, wherein the active ingredient(s) in the isotropic expanded microporous matrix are in the dissolved or dispersed state or in film-coated forms.

20. The composition of claim 19, wherein the active ingredient(s) are selected, without limitation, from analgesics, antimigraines, antipyretic analgesics and/or anti-inflammatory agents, local anesthetics, antianginals, anticholinergic antispasmodics, antisecretory agents, muscle relaxants, antinauseants, and central and peripheral vasodilators.

21. The composition of claim 20, wherein the active ingredient is selected from the group consisting of minalcipran, piroxicam, phloroglucinol and domperidone.
